(19) United States
(12) Patent Application Publication
(10) Pub. No.: US 2017/0007822 A1
(43) Pub. Date: Jan. 12, 2017

(54) ELECTROTHERAPY CABLE

(71) Applicant: U-GYM Technology Corporation, New Taipei City (TW)

(72) Inventors: Fu-Hao YU, New Taipei City (TW); Chin-Chuan HSU, New Taipei City (TW)

(21) Appl. No.: 15/203,369

(22) Filed: Jul. 6, 2016

(30) Foreign Application Priority Data
Jul. 8, 2015 (TW) 104211018

(51) Int. Cl. A61N 1/04

(52) U.S. Cl. CPC A61N 1/0488; A61N 1/0492

(57) ABSTRACT

An electrotherapy cable is installed with plural conductive patches and is constituted by a plug terminal, a cable line, an LED element and a conductive connector. Accordingly, the connection state of an electrotherapy machine can be known through the display state of the LED element, so as to facilitate identifying the usage state of the electrotherapy machine.

### BACKGROUND OF THE INVENTION

a) Field of the Invention

The present invention relates to an electrotherapy cable and more particularly to an electrotherapy cable, a usage state of which can be identified easily.

b) Description of the Prior Art

An ordinary electrotherapy machine needs to be connected with an electrotherapy while in use, and a tail end of the electrotherapy cable will be connected electrically with conductive patches. Therefore, when the electrotherapy machine is in use, the conductive patches can be attached on locations of body for electrotherapy. In order to allow the electrotherapy machine to be used in various intensities, the electrotherapy machine will be designed with plural intensity buttons to facilitate adjustment. However, when the electrotherapy machine is in use, a user is not able to know the usage state through the electrotherapy machine or other means, for example, whether the electrotherapy machine is energized to carry out the electrotherapy, whether the conduction state is good, or whether the connection state of the conductive wire is normal. Accordingly, whether the electrotherapy machine is in a good condition can be only determined based upon whether the electrotherapy machine is energized in use.

The detailed features and advantages of the present invention are described in details hereinafter, with the content being enough to make anyone familiar with the relevant skills to understand the technical content of the present invention and to implement accordingly. In accordance with the content, the claims and the drawings disclosed in this specification, any person skilled in the relevant art can easily understand the related objects and advantages of the present invention.

### SUMMARY OF THE INVENTION

The primary object of the present invention is to provide an electrotherapy cable, a connection state of which with an electrotherapy machine can be identified through the display of an LED (Light Emitting Diode) element, allowing a user to be aware of the usage state of the electrotherapy machine through the electrotherapy cable, which improves the convenience in using the electrotherapy machine.

To achieve the abovementioned object, the present invention discloses an electrotherapy cable which should be installed with plural conductive patches. The electrotherapy cable includes a plug terminal and a cable line. The plug terminal is provided with plural electrodes and these electrodes include a first electrode, a second electrode, a third electrode and a fourth electrode. An interior of the cable line is provided with plural conductive wires which are connected respectively with each electrode electrically, wherein the conductive wires connecting the first electrode and the third electrode are connected respectively with at least an LED element and an electrotherapy intensity regulator, electrically. In addition, the conductive wires connecting the second electrode and the fourth electrode are connected respectively with at least a conductive connector electrically, and each conductive connector is connected electrically with the conductive patch.

According to an embodiment of the present invention, the plug terminal is provided with a first blanket material for installing the LED element.

According to an embodiment of the present invention, each conductive connector is provided with a second blanket material for installing the LED element.

According to an embodiment of the present invention, the conductive connector is provided with a connection portion to combine with the conductive patch.

According to an embodiment of the present invention, the connection portion is a plug connector or a fastener.

According to an embodiment of the present invention, the second blanket material can be translucent material or a non-translucent material.

According to an embodiment of the present invention, the first blanket material can be translucent material or a non-translucent material.

According to an embodiment of the present invention, the conductive patch is provided with a butting portion to butt with the connection portion.

According to an embodiment of the present invention, the electrotherapy intensity regulator includes a circuit board, a first control module connecting the circuit board, and a second control module connecting the circuit board.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a three-dimensional schematic view of a preferred embodiment of the present invention.

FIG. 2 shows a schematic view of a usage state of the preferred embodiment of the present invention.

FIG. 3 shows a three-dimensional schematic view of another preferred embodiment of the present invention.

FIG. 4 shows a schematic view of a usage state of another preferred embodiment of the present invention.

### DETAILED DESCRIPTION OF THE PREFERRED EMBODIMENTS

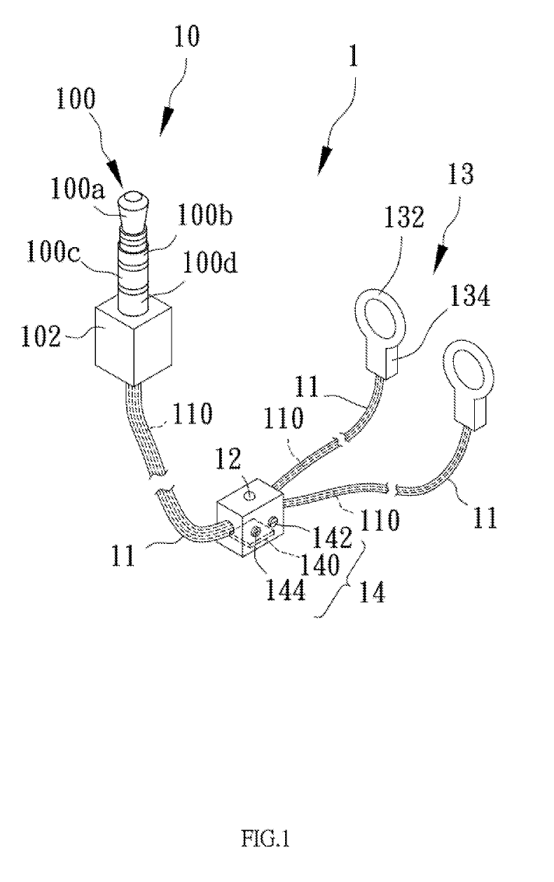

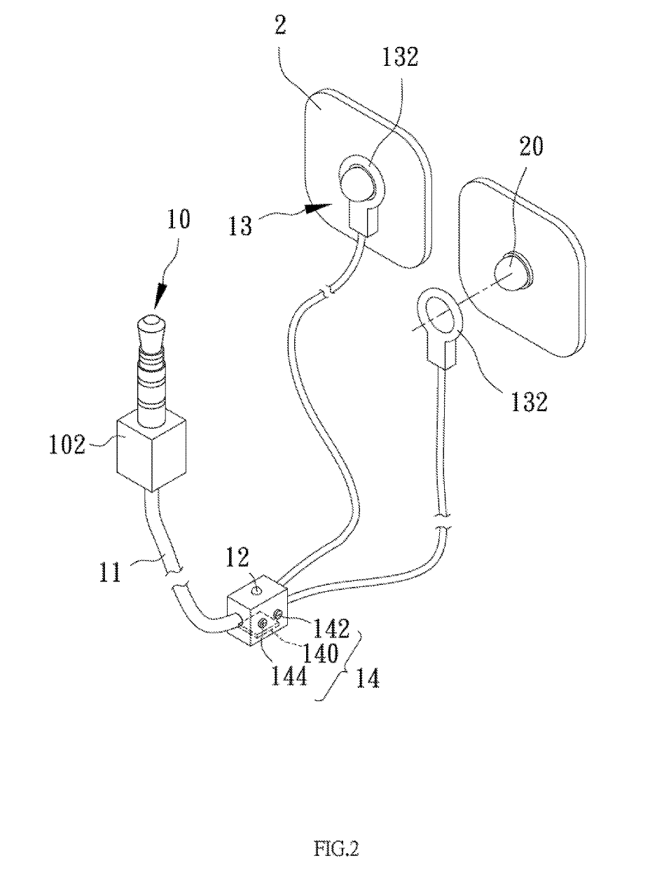

FIG. 1 and FIG. 2 show a three-dimensional schematic view and a schematic view of a usage state, of a preferred embodiment of the present invention. The present invention discloses primarily an electrotherapy cable 1 which should be installed with plural conductive patches 2 and be connected with an electrotherapy machine (not shown in the drawings) for electrotherapy. The electrotherapy cable 1 is constituted primarily by a plug terminal 10, a cable line 11, an LED element 12 and a conductive connector 13. The plug terminal 10 is provided with plural electrodes 100 which include a first electrode 100a, a second electrode 100b, a third electrode 100c and a fourth electrode 100d. In addition, an interior of the cable line 11 is provided with plural conductive wires 110 which are connected respectively with each electrode 100 electrically, wherein the conductive wires 110 connecting the first electrode 100a and the third electrode 100c are connected respectively with at least an LED element 12 and an electrotherapy intensity regulator 14, electrically. Furthermore, the conductive wires 110 connecting the second electrode 100b and the fourth electrode 100d are connected respectively with at least a conductive connector 13 electrically, and each conductive connector 13 is connected electrically with the conductive patch 2. As the conductive connector 13 is provided with a connection portion 132 (can be a plug connector or a fastener), the connection portion 132 can be engaged with a butting portion 20 disposed on the conductive patch 2 to energize electrically. On the other hand, the electrotherapy intensity regulator 14 includes a circuit board 140, a first control module 142 connecting the circuit board 140, and a second control module 144 connecting the circuit board 140.

The abovementioned plug terminal 10 should be enclosed with a first blanket material 102 (can be a translucent material or a non-translucent material), and the conductive connector 13 is enclosed with a second blanket material 134 (can be a translucent material or a non-translucent material). The LED element 12 can be installed in the first blanket material 102 or the second blanket material 134.

FIG. 2 shows a schematic view of a usage state of the preferred embodiment of the present invention. Referring to FIG. 1 and FIG. 2, in the present embodiment, the first blanket material 102 and the second blanket material 134 are a non-translucent material and the LED element 12 is exposed out of the first blanket material 102 and the second blanket material 134. The LED element 12 can be also disposed on the electrotherapy intensity regulator 14, and in the drawings, the LED element 12 is disposed on the electrotherapy intensity regulator 14, as an example. Before using the electrotherapy machine (not shown in the drawings), the plug terminal 10 should be inserted into a socket aperture of the electrotherapy machine, and each conductive connector 13 is connected electrically with the conductive patch 2. The connection portion 132 on the conductive connector 13 is a fastener as an example, and this fastener is primarily a closed loop. The butting portion 20 on the conductive patch 2 is a protruded latch point; therefore, the fastener can be latched tightly with the latch point. As the connection portion 132 and the butting portion 20 are made of metal, the effect of electricity conduction can be achieved. When the electric current is turned on in the electrotherapy machine, each electrode 100 in the plug terminal 10 will conduct the electric current, and the first electrode 100a, the second electrode 100b, the third electrode 100c and the fourth electrode 100d can allow the butted conductive wire 110 to transmit the electric current, enabling the LED element 12 to emit light and the conductive connector 13 to transmit the electric current to the conductive patch 2.

When the electrotherapy machine is used, the way that the LED element 12 emits light can be used to determine the usage state of the electrotherapy machine. In the present invention, there are three ways of emitting light to be used as a basis for determining the usage state of the electrotherapy machine. For the first way, the LED element 12 keeps emitting light always, representing that the electrotherapy machine is energized but the electrotherapy is not carried out. For the second way, the LED element 12 is flashing, meaning that the conduction is bad or the contact at the insertion hole is bad. For the third way, the LED element 12 gets dark from bright repeatedly, which means that the electrotherapy is ongoing.

Furthermore, the abovementioned electrotherapy intensity regulator 14 includes primarily a circuit board 140, a first control module 142 connecting the circuit board 140 and a second control module 144 connecting the circuit board 140. The first control module 142 is a press button to decrease the voltage, and the second control module 144 is a press button to increase the voltage. The user can adjust the intensity of the voltage through the first control module 142 and the second control module 144, via the circuit board 140.

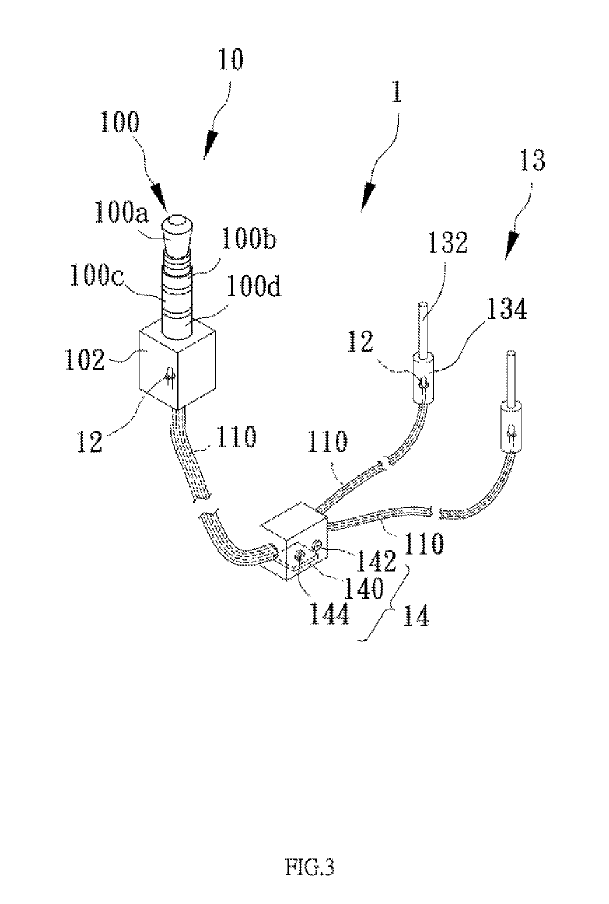

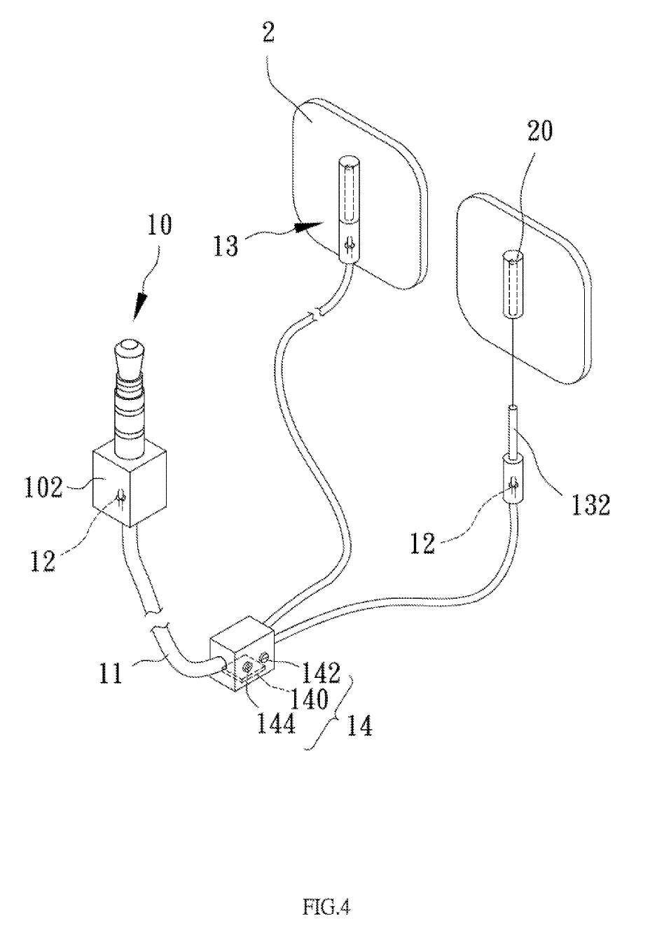

Referring to FIG. 3 and FIG. 4, it shows a three-dimensional schematic view and a schematic view of a usage state, of another preferred embodiment of the present invention. In this another preferred embodiment, the first blanket material 102 and the second blanket material 134 can be a translucent material, and the LED element 12 is disposed in the first blanket material 102 or the second blanket material 134. The user should insert the plug terminal 10 into the socket aperture of the electrotherapy machine (not shown in the drawings), and each conductive connector 13 is connected electrically with the conductive patch 2. The connection portion 132 on the conductive connector 13 is a plug connector, as an example, and this plug connector is primarily a cylinder. The butting portion 20 on the conductive patch 2, on the other hand, is a barrel. Therefore, the cylindrical connection portion 132 can be engaged tightly with the barrel-type butting portion 20. As the connection portion 132 and the butting portion 20 are made of metal, the effect of electricity conduction can be achieved. When the electric current is turned on in the electrotherapy machine, each electrode 100 in the plug terminal 10 will conduct the electric current, and the first electrode 100a, the second electrode 100b, the third electrode 100c and the fourth electrode 100d can allow the butted conductive wire 110 to transmit the electric current, enabling the LED element 12 to emit light and the conductive connector 13 to transmit the electric current to the conductive patch 2.

The connection state of the electrotherapy machine can be known through the display state of the LED element 12, allowing the user to identify the usage state of the electrotherapy machine through the electrotherapy cable 1, so as to improve the convenience in using the electrotherapy machine. It is naturally that the adjustment of the intensity of the voltage should be carried out with the electrotherapy intensity regulator 14. The electrotherapy intensity regulator 14 is constituted primarily by the circuit board 140, a first control module 142 connecting the circuit board 140 and a second control module 144 connecting the circuit board 140. The first control module 142 is a press button to decrease the voltage, and the second control module 144 is a press button to increase the voltage. The user can adjust the intensity of the voltage through the first control module 142 and the second control module 144, via the circuit board 140.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. An electrotherapy cable which should be installed with plural conductive patches, the electrotherapy cable comprising:
a plug terminal, which is provided with plural electrodes, the electrodes including a first electrode, a second electrode, a third electrode and a fourth electrode; and
a cable line, an interior of which is provided with plural conductive wires connecting respectively with each electrode electrically, with the conductive wires connecting the first electrode and the third electrode being connected respectively with an LED (Light Emitting Diode) element and an electrotherapy intensity regulator electrically, the conductive wires connecting the second electrode and the fourth electrode being connected with a conductive connector electrically, and each conductive connector being connected electrically with the conductive patch.

2. The electrotherapy cable according to claim 1, wherein the plug terminal is provided with a first blanket material for installing the LED element.

3. The electrotherapy cable according to claim 1, wherein each conductive connector is provided with a second blanket material for installing the LED element.

4. The electrotherapy cable according to claim 3, wherein the conductive connector is provided with a connection portion to be engaged with the conductive patch.

5. The electrotherapy cable according to claim 4, wherein the connection portion is a plug connector or a fastener.

6. The electrotherapy cable according to claim 5, wherein the second blanket material is a translucent material or a non-translucent material.

7. The electrotherapy cable according to claim 2, wherein the first blanket material is a translucent material or a non-translucent material.

8. The electrotherapy cable according to claim 4, wherein the conductive patch is provided with a butting portion to be butted with the connection portion.

9. The electrotherapy cable according to claim 1, wherein the electrotherapy intensity regulator includes a circuit board, a first control module connecting the circuit board, and a second control module connecting the circuit board.
